(19) United States
(12) Patent Application Publication
(10) Pub. No.: US 2011/0256167 A1
(43) Pub. Date: Oct. 20, 2011

(54) IMMUNOSTIMULATING SAPONINS FOR USE IN SITU TUMOR-DESTRUCTION THERAPY

(76) Inventors: Carla Christina Schrier, Boxmeer (NL); Stefan Nierkens, Nijmegen (NL); Gosse Adema, Nijmegen (NL); Martijn Den Brok, Nijmegen (NL); Theodoor Jaques Marie Ruers, Amsterdam (NL)

(21) Appl. No.: 13/141,157

(22) PCT Filed: Dec. 22, 2009
(86) PCT No.: PCT/EP09/67721

§ 371 Date: Jun. 21, 2011

(30) Foreign Application Priority Data
Dec. 23, 2008 (EP) 08172775.2

(51) Int. Cl. A61K 39/00; A61P 35/00

(52) U.S. Cl. 424/193.1; 424/277.1

(57) ABSTRACT

The present invention relates to pharmaceutical compositions for use in in situ tumor-destruction therapy comprising the steps of tumor destruction and administration of an immunostimulating amount of an immunopotentiator, and to the use of such pharmaceutical compositions in the manufacture of a medicament.

The present invention relates to pharmaceutical compositions for use in in situ tumor-destruction therapy comprising the steps of tumor destruction and administration of an immunostimulating amount of an immunopotentiator, and to the use of such pharmaceutical compositions in the manufacture of a medicament.

Cancer is a general term, used to describe neoplastic growth. Neoplasms are considered abnormal, usually de-differentiated forms of tissue that commonly proliferate at a higher speed than normal. In most cases neoplastic cells invade surrounding tissue and moreover they metastasize and continue to grow elsewhere in the body.

Local and regional treatment of the neoplastic mass, the tumor, such as surgery, does not affect possible metastases. Therefore, additional therapies are needed such as treatment with cytotoxic drugs. Such treatment is generally known as chemotherapy.

Local treatment is of course a first step in the treatment of solid tumors. This is traditionally done by means of tumor resection.

Another approach is tumor destruction in situ. A characteristic of tumor destruction in situ is that the tumor is not removed but necrotized. In principle irradiation is a form of tumor destruction in situ, but many other ways of tumor destruction have been developed. Common methods are e.g. photodynamic therapy using the combination of photosensitizing compounds and their subsequent activation by laser, in situ heating by means of laser light, microwaves, electric current, ultrasound, high intensity focused ultrasound or by means of radiofrequency waves, or cryotherapy: necrotizing tissue by freezing.

Tumor destruction in situ leaves the destructed tumor mass present in the body. This leaves the possibility open to try and build an immunological response to tumor-specific antigens (cancer immunotherapy). The advantage of successful induction of such an immunological response to tumor-specific antigens is that it will last for some time and eventually eliminate tumor localization elsewhere in the body that is not amenable for local tumor destruction.

However, contrary to what is known from vaccine development that is based upon non-self antigens, the induction of an immune response against tumor antigens is far from easy.

Basically, tumor antigens are predominantly normal components of the body: self-antigens. Therefore the immune system as such will down-regulate self-directed immune response leading to a tolerant state for self-antigens.

Thus, the development of tumor destruction-based cancer immunotherapy requires a very specific approach.

Actually, the non-methylated cytidyl guanosyl oligodeoxynucleotides (CpG ODN) are currently considered to be the by far most preferred specific group of immunopotentiating compounds capable of inducing an immune response against tumor-specific self-antigens.

These cytidyl guanosyl oligodeoxynucleotides act as toll-like receptor 9 (TLR9) agonists. CpG motifs stand out because of their preferential induction of Th1 responses and tumor-specific CD8⁺ T lymphocytes. TLR9 is predominantly expressed by B cells and dendritic cells (DC) that internalize and directly respond to CpG motifs. Upon triggering of TLR9, DCs mature and migrate to draining lymph nodes where they present antigens to T and B lymphocytes. Importantly, these DCs acquire the unique ability to present captured antigens on MHC class I molecules, a process known as cross-presentation, which is crucial for efficient priming of tumor-specific CTLs. As such, CpG administration has been reported to prevent tumor outgrowth in a prophylactic setting and could also eradicate established tumors in mice. Nierkens, S. et al. (Cancer Res. 68: 5390-5396 (2008)) and by Roux, S. et al. (Cancer Immunol Immunoth. 57: 1291-1300 (2008))

There are however some potential safety concerns with regard to the use of CpG ODNs, that i.a. include the induction of anti-DNA antibodies and autoimmunity. Furthermore, their toxicity when given in both higher amounts and over a longer period of time, as well as the costs involved in their use are of concern.

Therefore, there is a need for other immunopotentiating compounds.

The present invention provides means to decrease or overcome the concerns mentioned above.

Given the key role of TLR9 and its agonists in the induction of an immunological response to tumor-specific antigens after tumor destruction in situ, the skilled person would consider it a prerequisite that such other immunopotentiating compounds also act as TLR9 agonists.

Surprisingly it was found now that saponins, that bear no relation to the TLR9-mechanism at all, are nevertheless very suitable for inducing an immunological response to tumor-specific self-antigens after tumor destruction. Even more unexpectedly, their effectivity, although through unknown mechanisms, appears to be comparable to, or even better than that of CpGs.

It was found that the administration of saponins in or around a tumor, at or around the moment of tumor destruction induces a very significant immunological response to tumor-specific antigens after tumor destruction in situ. This immunological response is long-lasting and is therefore very suitable to eliminate metastasized cells, even if such cells have been latently present in the body. Moreover, this immunological response appeared to be sufficiently strong to prevent the multiplication of the same type of tumor cells even if these are deliberately administered in substantial amounts several weeks after the treatment.

Saponins have up till now only been described as adjuvants against non-self antigens; e.g. in bacterial or viral vaccines. The use of saponins as cytotoxins, for the killing of tumor cells, has been described by Bachran, C. et al. (Medicinal Chemistry 8: 575-584 (2008)). In PCT-application WO 2008/063129, the use of saponins in lipid-containing particles, as cytotoxins for the killing of tumor cells is described.

However in the present invention their cytotoxic effect is of no relevance, as the saponin is used in combination with cells that are already killed in the process of tumor destruction. Since their cytotoxic effect plays no role, no effect on the destructed tumor would be expected anyway. Moreover the cytotoxic effect of saponins in chemotherapy only works at the moment of administration. They do not build an immunological response and thus they do not act against metastasized cells that temporarily have low metabolic activity; latent cells.

The role of saponins in inducing an immunological response to tumor-specific self-antigens, following tumor destruction in situ was hitherto unknown and could for the reasons mentioned above not be expected.

Therefore, a first embodiment of the invention relates to pharmaceutical compositions for use in in situ tumor-destruction therapy comprising the steps of tumor destruction and administration of an immunostimulating amount of an immunopotentiator, wherein that said immunopotentiator is a saponin.

Saponin is in principle the generic name for a group of plant glycosides, of which the Quillaja saponaria saponin is the oldest and most frequently used.

Crude saponin actually is a mixture of saponins sharing a same basic structure, but having different side chains. The different saponin components differ mainly in their degree of hydrophilicity/phobicity.

HPLC is a preferred method to detect and isolate the various saponin components from a crude saponin mixture. Several purified extracts such as QS-7, −17, −18, −21, GPI-0100, QuilA, Qvac and BioQ are commercially available from various sources.

Preferably, saponin comprises at least one of the following components: QS-7, QS-17, QS-18 or QS-21.

Also preferred saponins are QuilA and components thereof, Vax Sap, SuperSap, GPI-0100, QP UF 1000 and the like.

Thus, a preferred form of this embodiment relates to a pharmaceutical composition according to the invention, wherein the saponin comprises at least one of the following components: QS-7, QS-17, QS-18, QS-21, QuilA, Vax Sap, SuperSap, GPI-0100 or QP UF 1000.

Another attractive immunostimulating form of saponin are so-called empty immune stimulating complexes (empty ISCOMS). Empty immune stimulating complex preparations differ from saponin in that they are made from a mixture of saponin, lipid and cholesterol. During their preparation small micelle-like particles are formed, that are even more immunopotentiating than saponin as such.

Thus, another preferred form of this embodiment relates to a pharmaceutical composition according to the invention wherein the saponin is in the form of an empty immune stimulating complex.

It goes without saying that the present invention is equally applicable in the field of human and veterinary medicine.

In view of the different modes of action of saponins on the one hand and CpG ODNs on the other hand, one could not expect any enhancing effect of the combined administration of the two.

Surprisingly, however, it was found that there is a strong synergistic effect in the combined use of saponin and CpG ODNs.

This unexpected synergy is advantageous, because this makes it possible to use sub-standard amounts of CpG ODNs when administered in combination with saponin. This in turn clearly diminishes the disadvantages of the use of CpG ODNs that were mentioned above. In this light, the use of CpG ODNs becomes attractive again, provided that they are given in combination with saponin.

The use of saponin in combination with CpG has been described as an adjuvant for the induction of an immune response against non-self antigens in U.S. Pat. No. 7,049,302, but for the reasons set out above a combined effect, let alone a synergistic effect, could be expected against self-antigens.

Thus, a more preferred form of this embodiment relates to a pharmaceutical composition according to the invention that in addition comprises CpG ODNs.

CpG ODNs for use in immune stimulation have been described since 1994 (U.S. Pat. No. 6,429,199). CpG-motifs basically have the structure 5′-X₁-C-pG-X₂-3′. The CpG motif 5′-Pu-Pu-CpG-Pyr-Pyr is known to be amongst the most immunopotentiating (Scheule, R. K., Advanced Drug Delivery Reviews 44: 119-134 (2000)). Basically, their length is from 8-80 bases and they contain at least one non-methylated CpG-motif.

Small differences in efficiency in different animal species are frequently seen. Merely as an example; human TLR9 is optimally triggered by the CpG motif G-T-CpG-T-T, whereas mouse TLR9 is more optimally triggered by G-A-CpG-T-T (Krieg, A. M., Nature Medicine 9: 831-835 (2003).

Optimal CpG motifs for seven veterinary and three laboratory species have been described by Rankin, R., et al., in Antisense and Nucleic Acid Drug Development 11: 333-340 (2001). CpG-motifs that efficiently stimulate canine and feline immune cell proliferation are described by Wernette, C. M., et al., in Veterinary Immunol And Immunopath. 84: 223-236 (2002). Applications for CpG-motifs in poultry have been described i.a. by Ameiss, K. A., et al., in Veterinary Immunol And Immunopath. 110: 257-267 (2006).

CpG ODNs with different CpG-motives are easily commercially available, and if desired they are easily synthesized. Suitable amounts of CpG ODNs can be found i.a. in the publications mentioned above and in the Examples section.

Again, as mentioned above, it goes without saying that the present invention is equally applicable in the field of human and veterinary medicine, although it is advisable (though not mandatory) to match the CpG-motif used to the animal species for which the invention is used. This can easily be done on the basis of the publications summarized above.

In principle, the steps of tumor destruction and administration of an immunopotentiator can be performed at different moments in time or at the same time. Theoretically, however, one would expect that conditioning a tumor with the immunopotentiator several days or better a week or even two or more weeks before applying tumor destruction, with the aim of “priming” the immune system, would be the preferred route.

Surprisingly however, it was found that if the administration of the immunopotentiator is done after tumor destruction, within days after tumor destruction, preferably within one day, more preferably within 12 hours, even more preferably within 6 hours, still even more preferably within 2 hours after tumor destruction, the level of immunostimulation is better than when the order of the steps is reversed.

Also very good results are obtained when the administration of the immunopotentiator is done between about two hours before the tumor destruction and the moment of destruction. This is because after destruction the neoplastic mass may be more difficult to approach or enter due to destruction-induced changes in its structure.

Administration of the immunopotentiator in the interval between 2 hours before and two hours after tumor destruction is called peri-operative administration.

Therefore, one preferred form of this embodiment relates to a pharmaceutical composition for use in in situ tumor-destruction therapy comprising the steps of tumor destruction and administration of an immunopotentiator according to the invention, wherein said steps are in the following order:
a. destruction of the tumor
b. administration of an immunopotentiator.

More preferred forms of this embodiment relate to the steps in the order as mentioned above wherein the administration of an immunopotentiator follows within 24 hours, 12 hours or even 6 hours after tumor destruction, in that order of preference.

Also, another preferred form of this embodiment relates to a pharmaceutical composition for use in in situ tumor-destruction therapy comprising the steps of tumor destruction and administration of an immunopotentiator according to the invention, wherein said steps are:
a. Peri-operative administration of the immunopotentiator
b. destruction of the tumor

With regard to the site or sites of administration of the immunopotentiator, the following considerations should be made:

Preferably, the immunopotentiator is administrated directly into the neoplastic mass. Although slightly less preferred, peri-tumoral administration where the immunopotentiator is administered at one or more locations around the neoplastic mass is also possible. Another, though less preferred administration is subcutaneous administration in the draining area of the neoplastic mass. Finally, intravenous administration, preferably close to the location of the neoplastic mass is possible.

Therefore, the said administration of the immunopotentiator takes place by intravenous administration, subcutaneous administration in the draining area of the neoplastic mass, peri-tumoral administration or intra-tumoral administration, in that order of increasing preference.

Another embodiment of the present invention relates to the use of a pharmaceutical composition according to the invention in the manufacture of a medicament for the treatment of a mammal suffering from cancer wherein the mammal has been subjected to tumor destruction.

Still another embodiment of the present invention relates to the use of a pharmaceutical composition according to the invention in the manufacture of a medicament for peri-operative administration, for the treatment of a mammal suffering from cancer wherein the mammal will be or has been subjected to tumor destruction.

### EXAMPLES

### Example 1

### Mice and Tumor Cells

C57BL/6n mice (6-8 weeks old) were purchased from Charles River Wiga (Sulzfeld, Germany) and maintained under specific pathogen-free barrier conditions at the Central Animal Laboratory (Nijmegen, The Netherlands). Drinking water and standard laboratory food pellets were provided ad libitum and mice were allowed to settle for at least 1 week before random assignment into specific treatment groups. The experiments were performed according to the guidelines for animal care of the Nijmegen Animal Experiments Committee.

The murine melanoma cell line B16F10 (ATCC) was cultured in complete medium (MEM, 5% fetal bovine serum (Greiner Bio-one), 100 U/ml penicillin G sodium and 100 μg/ml streptomycin (Pen/Strep), MEM sodium pyruvate (1 mM), NaHCO₃, MEM vitamins, MEM non-essential amino acids (all from Gibco), 20 μM β-mercaptoethanol (β-ME)).

Tumor Model and Cryosurgery

Tumor cells were suspended in a mixture of PBS and Matrigel (2:1), and 0.5*10⁶ cells in a total volume of 50 μl were injected s.c. at the right femur. When tumor diameters measured between 6-8 mm (generally at day 9-10) they were randomly assigned to treatment groups. Cryo ablation (Cryo) was performed under isoflurane/O₂/N₂O anesthesia using a liquid nitrogen cryoablation system (CS76, Frigitronics, Shelton, Conn.) of which the tip is cooled by a continuous flow of circulating liquid nitrogen. During 2 treatment cycles of freezing and thawing the tumor was macroscopically frozen, while leaving surrounding healthy tissue intact. To monitor the induction of long-lasting tumor protection, mice were re-challenged with 15*10³B160VA or B16F10 cells 40 days after cryo ablation. Re-challenges were injected in 100 μl PBS s.c. on the right flank. Mice were sacrificed when tumor volume exceeded 1000 mm³ or when tumors brake through the skin barrier.

### Adjuvant Injection

CpG 1668 ('5-TCCATGACGTTCCTGATGCT-3′) with total phosphorothioate-modified backbone was purchased from Sigma Genosys (Haverhill, UK). CpG was injected in PBS peri-tumorally (p.t., 30 μg divided over 2 injections of 10 μl lining the ablated tumor). The following adjuvants were used (all supplied by Intervet BV, Boxmeer): a water-in-oil emulsion based on mineral oil (Marcol 52) (1) and a water-in-oil emulsion based on non-mineral oil (Miglyol 840) (1); an oil-in-water emulsion using mineral oil, an oil-in-water emulsion using squalene (2); and an oil-in-water emulsion using vitamin E acetate (3); Matrix C 750 μg/ml (Isconova); Quil A Saponin (Brenntag) 500 μg/ml; aluminum hydroxide (Brenntag) 0.75% (w/v); or aluminum phosphate (Brenntag) 0.75% (w/v). In this article, the two water-in-oil emulsions were mixed at a 1:1 ratio and the three oil-in-water emulsions were mixed at a 1:1:1 ratio. The aluminum-based adjuvants were used mixed at a 1:1 ratio, but also separately. All non-microbial adjuvants (or mixes of them) were injected p.t. (40 μl divided over 2 injections of 20 μl, spatially separated from the CpG-ODN injections). All injections were done within 30 min. after ablation.

(1: Jansen et al, Vaccine, 23, 1053-1060, 2005, 2: O'Hagan Expert Re. Vaccines, 6, 669-710, 2007, 3: Rijke et al, in Adv. Avian Immunol Res. Eds. T. F. Davison, N. Bumstead and P. Kaiser 265-271, 1995)

### Statistical Analyses

Kaplan Meier survival curves were analyzed using a log rank test.

### Results:

As follows clearly from the graphs of FIG. 1, the combination of tumor destruction and the administration of CpG ODN as immunopotentiator leads to a sub-50% survival rate after 80 days. Moreover, there is no significant leveling off of the survival curve (FIG. 1a).

The combination of tumor destruction and the administration of the oil-in-water, the water-in-oil or the AlOH adjuvants as immunopotentiator all led to less protection (FIGS. 1b and 1c).

However, the combination of tumor destruction and the administration of saponin, be it in the form of QuilA or as empty immune stimulating complexes, as immunopotentiator leads to an impressive survival rate of >75% after 80 days. Moreover, in this case there is significant leveling off of the survival curve (FIG. 1c).

The combination of tumor destruction and the combined administration of CpG and saponin, be it in the form of QuilA or as empty immune stimulating complexes, as immunopotentiator leads to an even higher survival rate of >90% after 80 days and a very strong leveling off of the survival curve (FIG. 1d).

### LEGEND TO THE FIGURES

FIG. 1. Potent anti-tumor immunity following ablation combined with CpG-ODN and saponin-based adjuvants. Established B16F10 tumors on the right femur were treated with cryo ablation alone, in combination with CpG, or in combination with the indicated non-microbial adjuvants. Forty days later, naïve and tumor-free mice received a s.c. re-challenge with tumor cells (15.000 B16F10 cells) at the flank. The tumor size was monitored every 2-4 days.

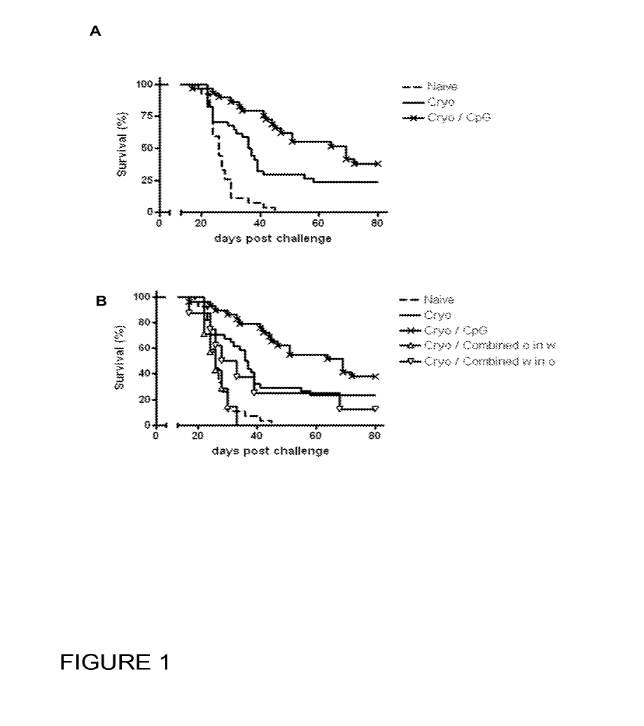

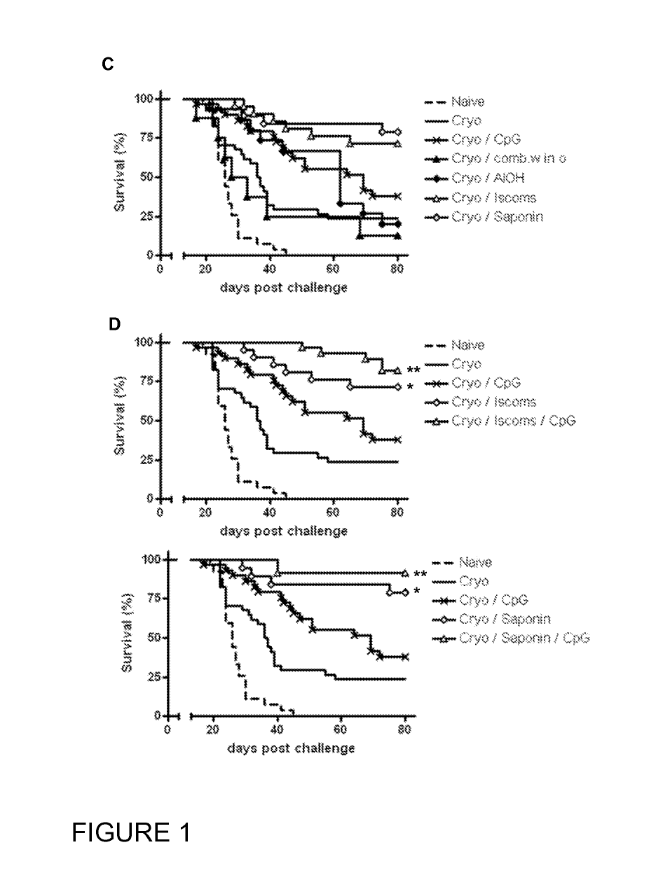

(A) Kaplan-Meier survival curve demonstrating limited protection from tumor outgrowth after ablation alone, or in combination with CpG-ODN.
(B) Survival curves demonstrating limited or no protection from tumor outgrowth after ablation alone, or in combination with the mixed oil-in-water, water-in-oil or aluminum adjuvants.
(C) Survival curve demonstrating relative protection from tumor outgrowth after ablation alone, or in combination with the indicated (mixed) adjuvants. The saponin-based adjuvants show the most potent protection.
(D) Survival curve demonstrating additional protection when ablation in combination with the saponin-based adjuvants is combined with CpG-ODN co-administration. *=p<0.05 compared to cryo, **=p<0.001 compared to cryo/CpG. Comparable data were obtained in three independent experiments.

1. A pharmaceutical composition for use in in situ tumor-destruction therapy comprising the steps of tumor destruction and administration of an immunostimulating amount of an immunopotentiator, characterized in that said immunopotentiator is a saponin.

2. The pharmaceutical composition according to claim 1, characterized in that said saponin comprises at least one of the following components: QS-7, QS-17, QS-18, QS-21, QuilA, Vax Sap, SuperSap, GPI-0100 or QP UF 1000.

3. The pharmaceutical composition according to claim 1, characterized in that the saponin is in the form of an empty immune stimulating complex.

4. The pharmaceutical composition according to claim 1, characterized in that in addition it comprises a CpG ODN.

5. The pharmaceutical composition for use in in situ tumor-destruction therapy comprising the steps of tumor destruction and administration of an immunopotentiator according to claim 1, characterized in that said steps are in the following order:
a. destruction of the tumor
b. administration of an immunopotentiator

6. The pharmaceutical composition according to claim 5, characterized in that said step of administration of an immunopotentiator follows within 24 hour after tumor destruction.

7. The pharmaceutical composition according to claim 5, characterized in that said step of administration of an immunopotentiator follows within 12 hour after tumor destruction.

8. The pharmaceutical composition according to claim 5, characterized in that said step of administration of an immunopotentiator follows within 6 hour after tumor destruction.

9. The pharmaceutical composition for use in in situ tumor-destruction therapy comprising the steps of tumor destruction and administration of an immunopotentiator according to claim 1, characterized in that said steps are:
a. peri-operative administration of an immunopotentiator
b. destruction of the tumor

10. The pharmaceutical composition according to claim 1, where the site of administration of the immunopotentiator is intravenous, subcutaneous in the draining area of the neoplastic mass, peri-tumoral or intra-tumoral, in that order of increasing preference.

11. A method of treating a mammal suffering from cancer comprising administering to the mammal the pharmaceutical composition according to claim 1 wherein the mammal has been subjected to tumor destruction.

12. A method of treating a mammal suffering from cancer comprising administering to the mammal the pharmaceutical composition according to claim 1, wherein said administering is performed as a peri-operative administration, wherein the mammal will be or has been subjected to tumor destruction.
